(19) United States
(12) Patent Application Publication
(10) Pub. No.: US 2026/0315669 A1
(43) Pub. Date: Oct. 15, 2026

(54) NEGATIVE PRESSURE ADSORPTION DEVICE

(71) Applicant: Reestar International Limited, Hong Kong (CN)

(72) Inventors: Geoffrey Koon Fu Hui, Hong Kong (CN); Wesley King Juen Tong, Hong Kong (CN)

(21) Appl. No.: 19/403,042

(22) Filed: Nov. 27, 2025

(30) Foreign Application Priority Data
Apr. 11, 2025 (CN) 202520686809.8

(51) Int. Cl. A61M 1/08

(52) U.S. Cl. CPC A61M 1/08; A61M 2202/0057; A61M 2205/3569; A61M 2205/3584

(57) ABSTRACT

A negative pressure adsorption device includes therapy assemblies. Each of the therapy assemblies includes a host, a working module, a control module, and a communication module. Each of the working module, the control module, and the communication module are mounted in the host. The control module is electrically connected to each of the working module and the communication module. The communication module of each of the therapy assemblies is configured to receive a user instruction. Or, the communication module of at least one of the therapy assemblies is configured to receive the user instruction, and the communication module of one of the therapy assemblies is communicatively connected to the communication module of another one of the therapy assemblies; wherein the therapy assemblies are capable of performing cupping operation synchronously on various parts of a user's body.

### CROSS REFERENCE TO RELATED APPLICATIONS

This application claims foreign priority of the Chinese patent application No. 202520686809.8, filed on Apr. 11, 2025. The disclosure of which is hereby incorporated by reference in their entireties.

### FIELD OF THE DISCLOSURE

The present disclosure relates to the technical field of negative pressure adsorption, and particularly to a negative pressure adsorption device.

### BACKGROUND

Cupping, as a method of external treatment in traditional Chinese medicine, utilizes combustion to remove air from a cup, creating negative pressure that causes the cup to adsorb on specific parts of body surface, such as meridian points or painful areas. Through local skin reactions like congestion and bruising, it functions to unblock meridians, promote qi and blood circulation, reduce swelling and relieve pain, dispel wind and cold, etc., aiming to regulate body functions and prevent/treat diseases. However, a method of exhausting air by combustion requires ignition, which is unsafe. Some body parts are reachable by hand, but due to a need for ignition and combustion for adsorption, individuals cannot perform the cupping themselves.

When using a single electric negative pressure adsorption device, the electric negative pressure adsorption device can only be adsorbed on corresponding parts sequentially for treatment, or multiple negative pressure adsorption devices can be used, each adsorbed on a corresponding part. Each negative pressure adsorption device requires individual adjustment, making a usage process time-consuming, laborious, inefficient, operationally limited, and cumbersome.

### SUMMARY

The following presents a simplified summary of the disclosure in order to provide a basic understanding of some aspects of the disclosure. This summary is not an extensive overview of the disclosure. It is not intended to identify critical elements or to delineate the scope of the disclosure. Its sole purpose is to present some concepts of the disclosure in a simplified form as a prelude to the more detailed description that is presented elsewhere.

In some embodiments, a negative pressure adsorption device is provided. The negative pressure adsorption device includes at least two therapy assemblies. Each of the at least two therapy assemblies includes a host, a working module, a control module, and a communication module. The host includes a body portion and a cup portion connected with the body portion. The body portion defines an accommodation cavity. Each of the working module, the control module, and the communication module are mounted in the accommodation cavity. The control module is electrically connected to each of the working module and the communication module. The working module is configured to form a negative pressure in the cup portion. The communication module of each of the at least two therapy assemblies is configured to receive a user instruction. Or, the communication module of at least one of the at least two therapy assemblies is configured to receive the user instruction, and the communication module of one of the at least two therapy assemblies is communicatively connected to the communication module of another one of the at least two therapy assemblies. The at least two therapy assemblies are capable of performing cupping operation synchronously on various parts of a user's body.

### BRIEF DESCRIPTION OF THE DRAWINGS

Illustrative embodiments of the present disclosure are described in detail below with reference to the attached drawing figures.

FIG. 1 is a schematic view of an external structure of a therapy assembly according to an embodiment of the present disclosure.

FIG. 2 is a cross-sectional view of the therapy assembly according to an embodiment of the present disclosure.

FIG. 3 is an exploded view of a partial structure of the therapy assembly according to an embodiment of the present disclosure.

FIG. 4 is an exploded view of the therapy assembly according to an embodiment of the present disclosure.

FIG. 5 is a structural view of a cup portion of the therapy assembly according to an embodiment of the present disclosure.

FIG. 6 is a structural view of another cup portion of the therapy assembly according to an embodiment of the present disclosure.

FIG. 7 is another exploded view of the therapy assembly according to an embodiment of the present disclosure.

FIG. 8 is another exploded view of the partial structure of the therapy assembly according to an embodiment of the present disclosure.

FIG. 9 is a block diagram of a relationship between a plurality of therapy assemblies according to an embodiment of the present disclosure.

FIG. 10 is a block diagram of the relationship between the plurality of therapy assemblies according to another embodiment of the present disclosure.

FIG. 11 is a block diagram of the relationship between the plurality of therapy assemblies according to yet another embodiment of the present disclosure.

FIG. 12 is a block diagram of the therapy assembly with an instruction module implemented as a remote controller according to an embodiment of the present disclosure.

FIG. 13 is a block diagram of the therapy assembly with the instruction module implemented as a button module according to an embodiment of the present disclosure.

FIG. 14 is a block diagram of the therapy assembly with the instruction module implemented as a voice module according to an embodiment of the present disclosure.

FIG. 15 is a block diagram of the therapy assembly with three instruction modules implemented as a remote controller, a button module, and a voice module respectively according to an embodiment of the present disclosure.

FIG. 16 is a block diagram of the therapy assembly where a user instruction is issued by a smart terminal according to an embodiment of the present disclosure.

FIG. 17 is a block diagram of a negative pressure adsorption device in Embodiment I.

FIG. 18 is a block diagram of the negative pressure adsorption device in Embodiment II.

FIG. 19 is a block diagram of the negative pressure adsorption device in Embodiment III.

FIG. 20 is a block diagram of the negative pressure adsorption device in Embodiment IV.

FIG. 21 is a block diagram of the negative pressure adsorption device in Embodiment V.

### NUMERAL REFERENCE IN THE DRAWINGS

10—therapy assembly; 11—host; 12—working module; 13—control module; 14—communication module; 15—built-in power supply; 121—negative pressure unit; 122—heating unit; 131—timing unit; 101—master therapy assembly; 102—slave therapy assembly; 111—body portion; 112—cup portion; 113—foam filter; 114—metal contact piece; 1111—cover; 1112—housing; 111a—accommodation cavity; 1221—infrared emitter; 1222—light—transmitting cover; 1111a—mounting hole; 1112a—negative pressure hole;
20—instruction module; 21—remote controller; 22—button module; 23—voice module;
30—smart terminal;
200—user instruction.

### DETAILED DESCRIPTION

The following describes some non-limiting exemplary embodiments of the disclosure with reference to the accompanying drawings. The described embodiments are merely a part rather than all of the embodiments of the disclosure. All other embodiments obtained by a person of ordinary skill in the art based on the embodiments of the disclosure shall fall within the scope of the disclosure.

Unless otherwise defined, all technical and scientific terms used herein have the same meaning as commonly understood by one of ordinary skill in the art to which this invention belongs; the terminology used in the description of the application herein is for the purpose of describing particular embodiments only and is not intended to be limiting of the invention; the terms “comprising” and “having” and any variations thereof in the description and claims and the above description of the accompanying drawings are intended to cover non-exclusive inclusions. The terms “first,” “second,” etc. in the description and claims or the above accompanying drawings are used to distinguish different objects, not to describe a specific order.

References herein to “embodiment” mean that a particular feature, structure, or characteristic described in connection with the embodiment may be included in at least one embodiment of the invention. The appearance of the phrase in various places in the specification does not necessarily all refer to the same embodiment, nor are they independent or alternative embodiments mutually exclusive of other embodiments. Those skilled in the art will explicitly and implicitly understand that the embodiments described herein may be combined with other embodiments.

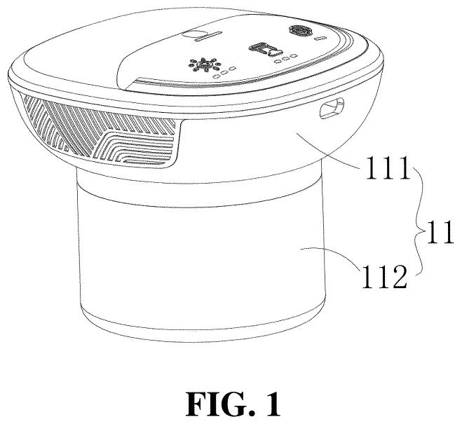

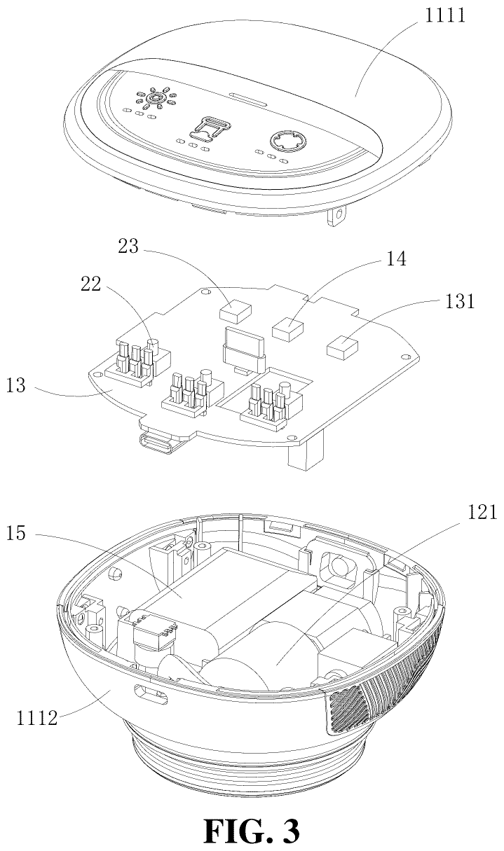

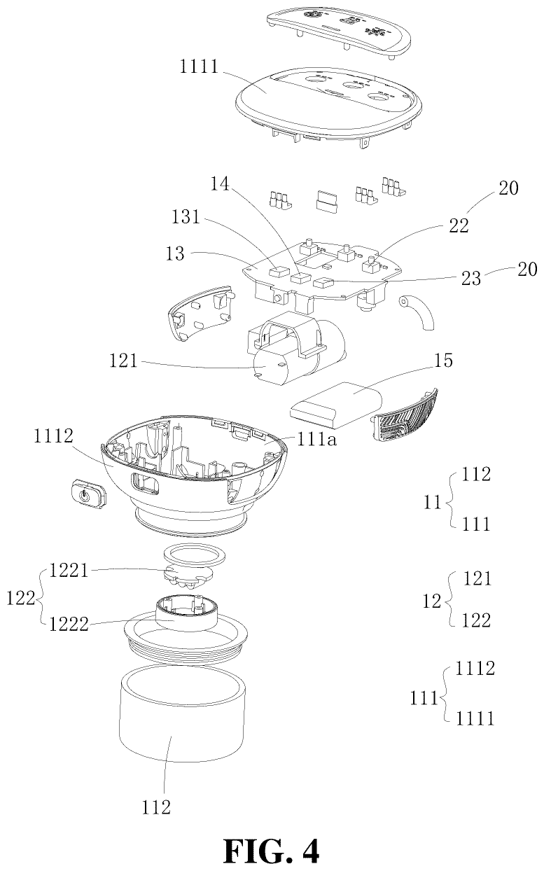

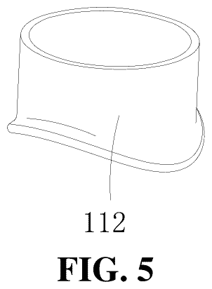

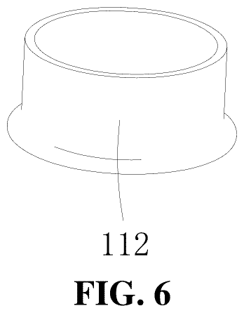

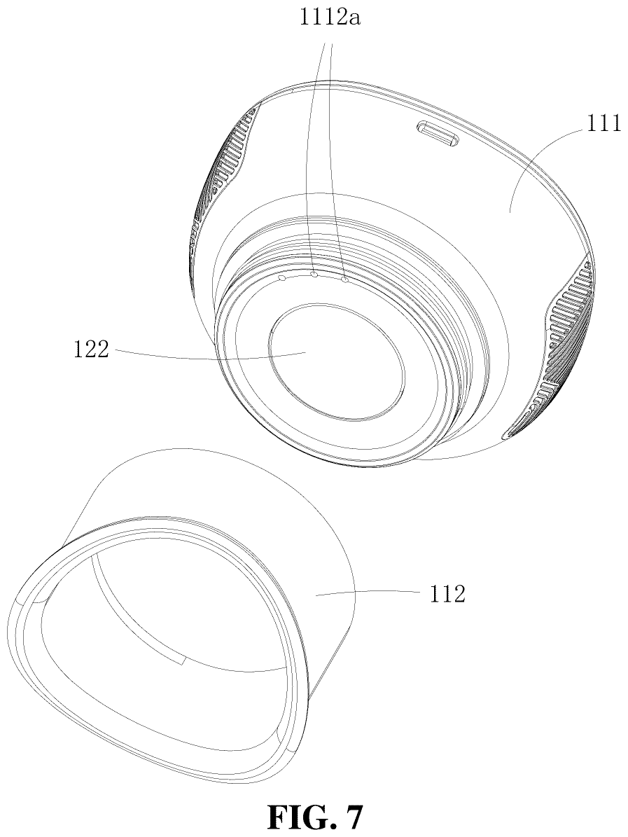

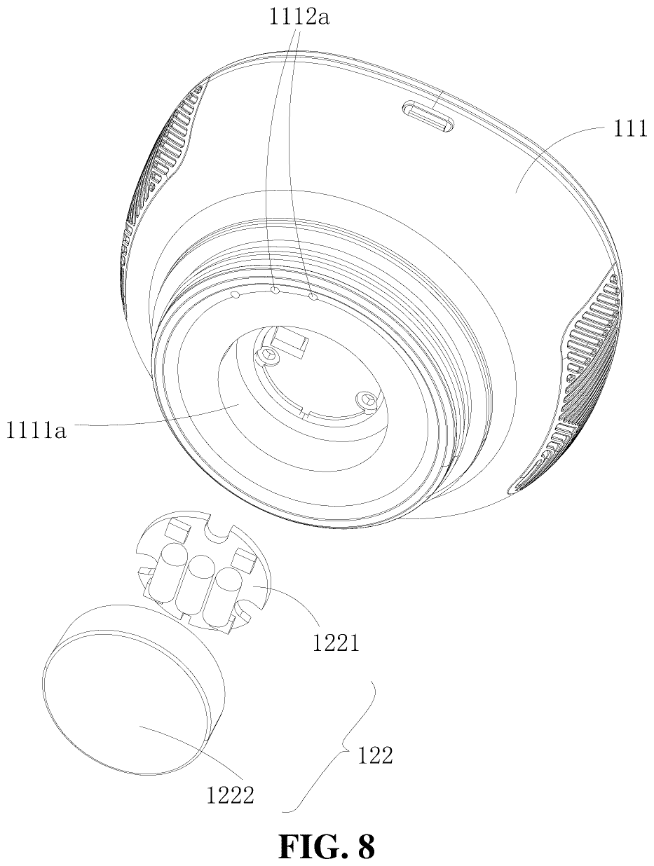

Referring to FIGS. 1 to 21, the present disclosure provides a negative pressure adsorption device. The negative pressure adsorption device may include at least two therapy assemblies 10. Each therapy assembly 10 may include a host 11, a working module 12, a control module 13, and a communication module 14. Each of the working module 12, the control module 13, and the communication module 14 may be mounted in the host 11. The control module 13 may be electrically connected to each of the working module 12 and the communication module 14. The control module 13 may be a circuit board. The host 11 may include a body portion 111 and a cup portion 112 arranged on the body portion 111. Each of the control module 13, the communication module 14, and the working module 12 may be arranged in the body portion 111. The cup portion 112 may be detachably connected to the body portion 111. The working module 12 may form a negative pressure in the cup portion 112 and enable the cup portion 112 to adsorb on human skin. The body portion 111 may be arranged with a built-in power supply 15. A shape of a cup mouth of the cup portion 112 may be arranged in various styles according to different parts of human body, such as an arc-shaped cup mouth adapted to human knees, shoulders, etc., as shown in FIG. 5, or a straight cup mouth suitable for adsorption on a back or abdomen, as shown in FIG. 6.

The body portion 111 may define an accommodation cavity 111a. Each of the control module 13, the communication module 14, and the working module 12 may be arranged in the accommodation cavity 111a. The body portion 111 may include a cover 1111 and a housing 1112. One of two opposite ends of the housing 1112 may be connected to the cover 1111. The cover 1111 and the housing 1112 may cooperatively define the accommodation cavity 111a. The working module 12, the control module 13, the communication module 14, and the built-in power supply 15 may be arranged in the accommodation cavity 111a. The other of the two opposite ends of the housing 1112 may be connected to the cup portion 112, specifically through a threaded connection to achieve a detachable connection between the housing 1112 and the cup portion 112, facilitating replacement of the cup portion 112.

As the cup portion 112 may be made of glass and prone to breakage, to solve this problem, the body portion 111 may be arranged with a grip portion for easy holding. The body portion 111 may be arranged in a shape of a truncated cone that is larger at a top portion and smaller at a bottom portion. In other words, a diameter of a side of the body portion 111 close to the cup portion 112 is smaller than a diameter of a side of the body portion 111 away from the cup portion 112. A side of the body portion 111 extending from the bottom portion of the body portion 111 to the top portion of the body portion 111 may serve as the grip portion. A shape of the grip portion which may be larger at a top portion and smaller at a bottom portion may make grip of the host 11 more secure. An outermost layer of the grip portion may be made of a soft rubber material. The soft rubber material may exhibit a high damping coefficient and may enhance an anti-slip effect of the grip portion. In addition, the outermost layer of the grip portion may be arranged with raised anti-slip structures to further strengthen the anti-slip effect.

The working module 12 may be used to perform negative pressure adsorption work, such as for cupping. The working module 12 may include a negative pressure unit 121 and a heating unit 122. The negative pressure unit 121 may generate a negative pressure required for cupping, creating a vacuum environment in the host 11, thereby enabling the therapy assembly 10 to adsorb on the skin. The negative pressure unit 121 may be a pump, and a suction intensity of the negative pressure unit 121 may be adjusted, for example, by setting a plurality of adjustment levels. Correspondingly, one or more negative pressure holes 1112a may be formed in a portion of the housing 1112 connected with the cup portion 112 to communicate the negative pressure unit 121 with a cavity defined by the cup portion 112. This allows the negative pressure generated by the negative pressure unit 121 to be transmitted through the cup portion 112 to an area of the human body requiring therapy. Specifically, the one or more negative pressure holes 1112a may be formed at an end of the housing 1112 away from the cover 1111, i.e., facing the cup portion 112, and may be distributed at a periphery of the heating unit 122. Furthermore, a foam filter 113 may be arranged between the cup portion 112 and the body portion 111 to prevent substances such as dust, dead skin, and oil from entering the pump during a suction process, preventing pump damage. The heating unit 122 may heat an interior of the host 11, helping to promote blood circulation and improve a cupping effect. The heating unit 122 may include one or a combination of an electric heating wire, a heating sheet, and an infrared laser emitter. The infrared laser emitter may emit near-infrared or far-infrared light. The infrared laser emitter may be used for auxiliary physical therapy, as the infrared laser emitter may stimulate human tissue, promote blood circulation, relieve pain, accelerate wound healing, etc. For example, in an example, the end of the housing 1112 away from the cover 1111 may define a mounting hole 1111a. The heating unit 122 may be mounted in the mounting hole 1111a, arranged at the end of the housing 1112 away from the cover 1111, and may include an infrared emitter 1221 and a light-transmitting cover 1222. Specifically, the light-transmitting cover 1222 may be mounted surrounding a periphery of the infrared emitter 1221, with at least a portion of the light-transmitting cover 1222 located on a side of the infrared emitter 1221 away from the cover 1111, i.e., facing the cup portion 112, so as to transmit the infrared light emitted by the infrared emitter 1221 toward the cup portion 112. In this example, the communication holes 1112a may be formed surrounding a periphery of the heating unit 122. For example, there may be a plurality of communication holes 1112a, distributed surrounding an outer periphery of the heating unit 122.

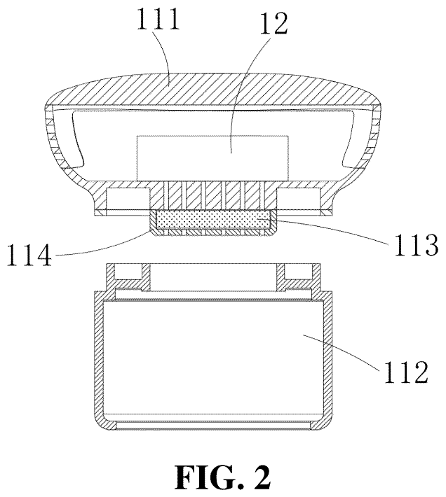

The working module 12 may also include a temperature control unit (heating/cooling unit) mounted in the body portion 111. The cooling unit and the heating unit 122 may be formed as a same component. Referring to FIG. 2, the body portion 111 may be arranged with a metal contact piece 114. The metal contact piece 114 may be fixed or detachably mounted at an end of the body portion 111 where the body portion 11 is connected to the cup portion 112. The temperature control unit 122 may heat or cool the metal contact piece 114. When using the metal contact piece 114, the negative pressure unit 121 may not be used, and the cup portion 112 may be removed, allowing the metal contact piece 114 to directly contact the human skin and to heat or cool the human skin through the metal contact piece 114. Furthermore, regarding a heat dissipation issue in the body portion 111, in some embodiments, a heat dissipation fan may be mounted in the body portion 111, and heat dissipation holes may be formed in the host 11. In other embodiments, by optimizing a heat dissipation structure of the body portion 111, the body portion 111 may become a huge heat sink without a need for an additional heat dissipation fan. Furthermore, the metal contact piece 114 may be a porous structure, and the foam filter 113 may be arranged close to an inner side of the metal contact piece 114 to facilitate the negative pressure unit 121 in the body portion 111 forming a negative pressure in the cup portion 112. It should be noted that the inner side of the metal contact piece 114 is a side of the metal contact piece 114 facing the accommodation cavity 111a, i.e., away from the cup portion 112.

The control module 13 may be a control circuit board. The communication module 14 may be arranged on the control circuit board, specifically on a side of the control circuit board facing the cover 1111, i.e., away from the cup portion 112. The working module 12 (such as the negative pressure unit 121 and the heating unit 122) and the built-in power supply 15 may be located on a side of the control circuit board facing the cup portion 112.

The negative pressure suction function, cooling function, and heating function of the therapy assembly 10 may be used to relax user muscles, help relieve spasms, tension, and inflammation, and cooperate with the negative pressure unit 121 to eliminate waste from the user's body, improve blood circulation, and promote the user's physical recovery.

Regarding a synchronous control method for working modules 12 of a plurality of therapy assemblies 10, one of the following situations may be adopted.

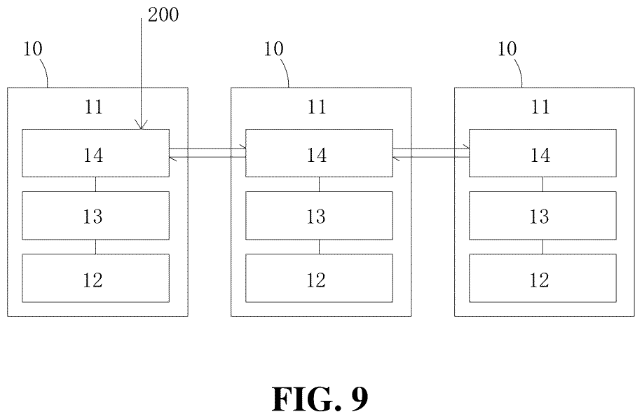

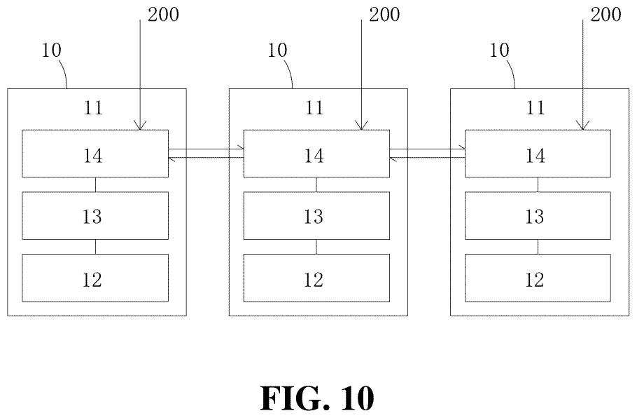

In some embodiments, the communication module 14 of at least one therapy assembly 10 may be enabled to receive a user instruction, and the communication module 14 of the at least one therapy assembly 10 may be communicatively connected with the communication module 14 of each of other therapy assemblies 10. The user instruction may contain at least information about a working number and working time of the working module 12. Therefore, when the communication module 14 of one therapy assembly 10 obtains the user instruction, an information setting for the control module 13 in a corresponding therapy assembly 10 may be completed, enabling the control module 13 to control the corresponding working module 12 to operate according to the user instruction. Simultaneously, the communication module 14 of the corresponding therapy assembly 10 may also send the received user instruction information to other therapy assemblies 10, facilitating the user's synchronous control of the plurality of therapy assemblies 10. For example, as shown in FIG. 9, only the communication module 14 of one therapy assembly 10 may be allowed to receive the user instruction, and then this corresponding therapy assembly 10 may share instruction information with other therapy assemblies 10. In this way, the user may only need to perform an operate setting on a specific therapy assembly 10, while other therapy assemblies 10 may synchronously complete corresponding operation settings. Or, for example, as shown in FIG. 10, any communication module 14 may receive the user instruction, and each therapy assembly 10 may share instruction information with other therapy assemblies 10. In this way, the user may perform the operation setting on any therapy assembly 10, and other therapy assemblies 10 may also synchronously complete the operation settings correspondingly.

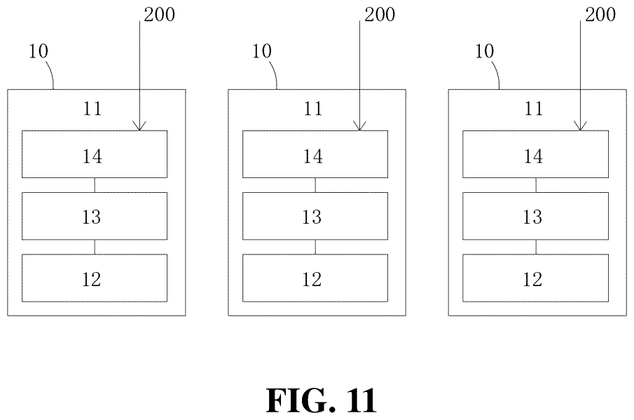

In other embodiments, for example, as shown in FIG. 11, the communication module 14 of each of the plurality of therapy assemblies 10 may receive a same user instruction, and the communication modules 14 of the plurality of therapy assemblies 10 may not need to interact with each other and may be arranged independently from each other. That is, the communication modules 14 of the plurality of therapy assemblies 10 may simultaneously receive the user instruction, so that the control module 13 of each therapy assembly 10 may only need to be performed an own information setting, also achieving synchronous control and setting of the plurality of therapy assemblies 10.

Therefore, the user may only need to perform a mode setting operation once to make all of the plurality of therapy assemblies 10 complete a mode setting, without a need to repeatedly operate the plurality of therapy assemblies 10 one by one. That is, a synchronous control of the plurality of therapy assemblies 10 may be achieved, quickly completing a series of settings for the plurality of therapy assemblies 10, improving convenience.

Regarding a source of the user instruction, at least one instruction module 20 may be optionally arranged, and the user may send the instruction information to the therapy assembly 10 by operating the instruction module 20. Or, the instruction information may be sent to the therapy assembly 10 by a smart terminal 30. Specific examples are given in the following embodiments.

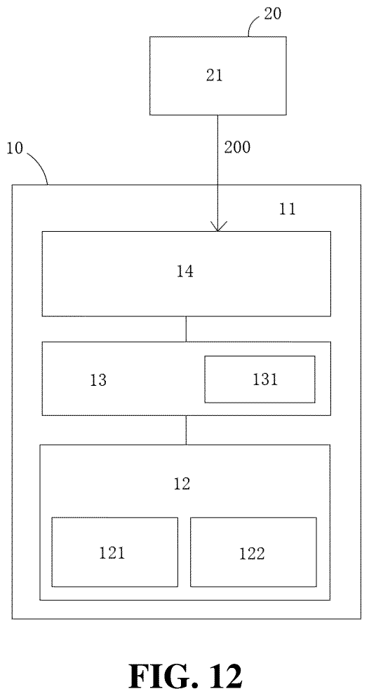

In some embodiments, for example, as shown in FIG. 12, the negative pressure adsorption device may include at least one instruction module 20. One of the at least one instruction module 20 may be a remote controller 21. The remote controller 21 may be signally connected to the communication module 14, allowing the user to operate the negative pressure adsorption device from a certain distance.

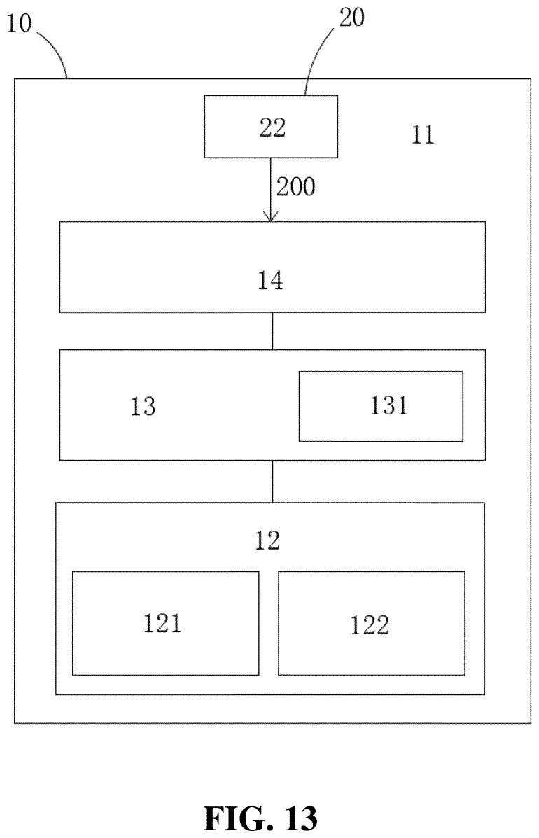

In other embodiments, for example, as shown in FIG. 13, the negative pressure adsorption device may include at least one instruction module 20. One instruction module 20 may be a button module 22 mounted on a corresponding host 11. The button module 22 may be mounted on the control module 13, i.e., the control circuit board, and located on the side of the control circuit board facing the cover 1111, i.e., away from the cup portion 112. The button module 22 may be electrically connected to the communication module 14 in the corresponding host 11 and the control module 13 in the corresponding host 11. The user may send an electrical signal to the communication module 14 and the control module 13 by pressing the button module 22. In other words, the user may operate directly on a certain therapy assembly 10. The button module 22 may be a circuit board and a button integrated on the circuit board.

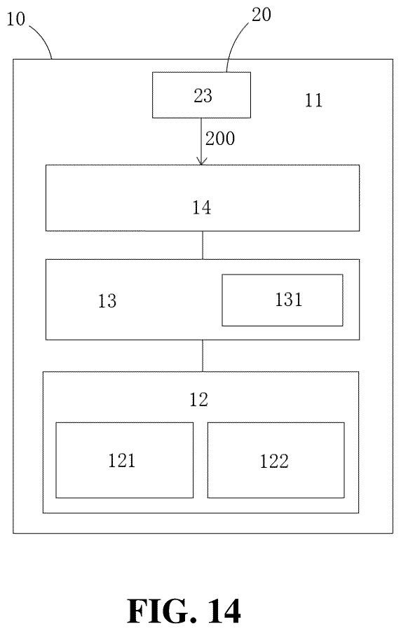

In further embodiments, for example, as shown in FIG. 14, the negative pressure adsorption device may include at least one instruction module 20. One instruction module 20 may be a voice module 23 mounted on a corresponding host 11. The voice module 23 may be mounted on the control module 13, i.e., the control circuit board, and located on the side of the control circuit board facing the cover 1111, i.e., away from the cup portion 112. The voice module 23 may be electrically connected to the communication module 14 in the corresponding host 11 and the control module 13 in the corresponding host 11. The user's voice may be converted to the user instruction by the voice module 23 and sent to the control module 13 and the communication module 14, meaning the user may not need to manually control the negative pressure adsorption device, which may improve the convenience of user operation.

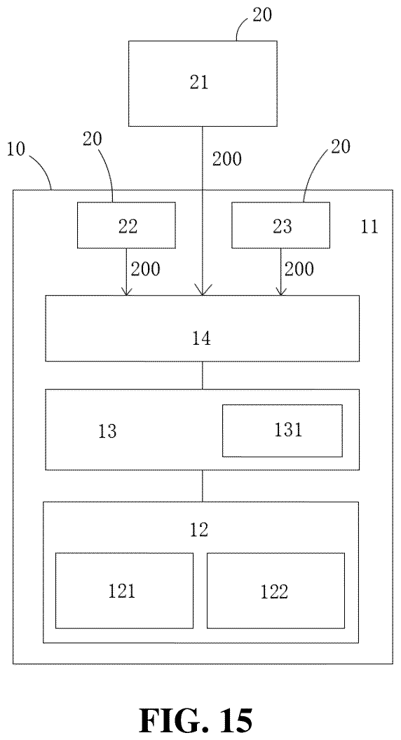

In yet other embodiments, for example, as shown in FIG. 15, the negative pressure adsorption device may include at least three instruction modules 20. Three of the instruction modules 20 may be a remote controller 21, a button module 22, and a voice module 23, respectively. An arrangement of each of the remote controller 21, the button module 22, and the voice module 23 in this embodiment may refer to the previous three embodiments. That is, this embodiment may combine control methods of the previous three embodiments, allowing the user to choose to operate any one of the instruction modules 20 to complete settings for each therapy assembly 10.

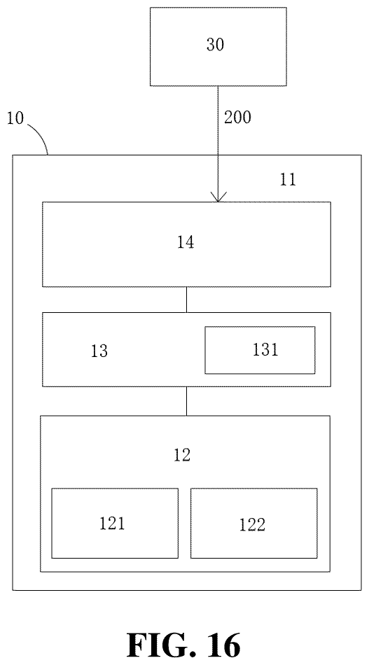

In other embodiments, for example, as shown in FIG. 16, a smart terminal 30 may serve as a signal transmitter. The smart terminal 30 may be, for example, a mobile phone, tablet computer, etc. An application may be installed on the smart terminal 30, and the user may operate the application to make the smart terminal 30 emit specific signals. In this case, the smart terminal 30 may not be part of the negative pressure adsorption device. It may only be necessary that at least one therapy assembly 10 in the negative pressure adsorption device may have a function of receiving specific signals, meaning a source of the user instruction may not be limited.

A communication connection method between the plurality of communication modules 14, i.e., an information sharing manner, may include at least one of Bluetooth connection, Wi-Fi connection, Zigbee, or RFID, or may be other wireless connection manner. These communication manners may meet needs of different usage scenarios. For example, Bluetooth may be suitable for short-distance, low-power communication between devices; Wi-Fi may be suitable for larger areas requiring higher data transmission rates; Zigbee may have low power consumption and self-organizing network characteristics; RFID may be used for non-contact data transmission.

In some embodiments, the therapy assembly 10 may further include a timing unit 131 mounted in the host 11. The timing unit 131 may be mounted on the control module 13, i.e., the control circuit board, and located on the side of the control circuit board facing the cover 1111, i.e., away from the cup portion 112. The timing unit 131 may be electrically connected to the working module 12 and the control module 13, used to determine various operational timings, such as a start time, a negative pressure suction time, and a heating time of the working module 12.

In some embodiments, the instruction module 20 may be preset with a plurality of preset user instructions. The preset user instructions may be associated at least with a working number and a working time of at least one therapy assembly 10, and may also be associated with information such as the suction intensity of the negative pressure unit 121, a heating temperature of the heating unit 122, and a working sequence. That is, the preset user instructions may contain work information such as “multi-cup linkage”, “time-sharing start/stop”, “pressure gradient adjustment”, etc., which may be called with one click by the user through the instruction module 20.

Each preset user instruction may be set according to different user needs. For example, for Role A, three therapy assemblies 10 may be set to work simultaneously, a total working time may be set to half an hour, the suction intensity of the negative pressure unit 121 may be set to level one, and the heating temperature of the heating unit 122 may be set to level two. For example, for Role B, four therapy assemblies 10 may be set to work simultaneously, the total working time may be set to fifteen minutes, the suction intensity of the negative pressure unit 121 may be set to level two, and the heating temperature of the heating unit 122 may be set to level three.

Furthermore, the instruction module 20 and the control module 13 may be allowed to automatically store a last selected work mode, equivalent to automatically generating the preset user instruction. When the negative pressure adsorption device is started again, the negative pressure adsorption device may quickly enter a specified work mode.

An operation method of the negative pressure adsorption device may be as follows.

When the user issues a start instruction through the instruction module 20 (such as the remote controller 21 or button module 22) or via the smart terminal 30, the instruction module 20 may transmit the start instruction to the communication module 14 of a corresponding therapy assembly 10. Upon the communication module 14 receives the start instruction, the communication module 14 may forward the start instruction to the control module 13, and may also transmit the start instruction to the communication modules 14 of other therapy assemblies 10 via a wireless signal. Based on information in the start instruction, the control modules 13 in a designated number of therapy assemblies 10 may start the corresponding negative pressure units 121 to begin generating negative pressure. When a delayed start of the negative pressure unit 121 is required, the timing unit 131 may start timing and the negative pressure unit 121 may start after reaching a preset time. Simultaneously, when a heating function is needed, the heating unit 122 may also be activated. Once the negative pressure unit 121 starts operating, the timing unit 131 may also begin timing synchronously. When the preset working time is reached, the control module 13 may stop both the negative pressure unit 121 and the heating unit 122, ensuring that the designated number of therapy assemblies 10 may work or stop synchronously.

Combining the above embodiments, at least the following embodiments may be obtained.

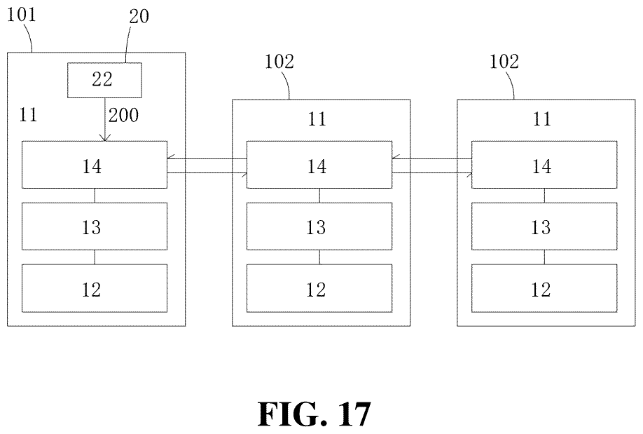

Referring to FIG. 17, in Embodiment I, the button module 22 may only be arranged on the host 11 of one therapy assembly 10. This one therapy assembly 10 may be named a master therapy assembly 101, and remaining therapy assemblies 10 may be slave therapy assemblies 102. The button module 22 on the master therapy assembly 101 may only be electrically connected to the communication module 14 and the control module 13 in the master therapy assembly 101 to transmit the user instruction. After the user presses several designated buttons, a work mode setting for the master therapy assembly 101 may be completed. Then, the communication module 14 of the master therapy assembly 101 may transmit the user instruction to other slave therapy assemblies 102 via the wireless signal, causing the other slave therapy assemblies 102 to also complete the work mode setting. That is, the slave therapy assemblies 102 may only obtain the user instruction through information interaction with the master therapy assembly 101.

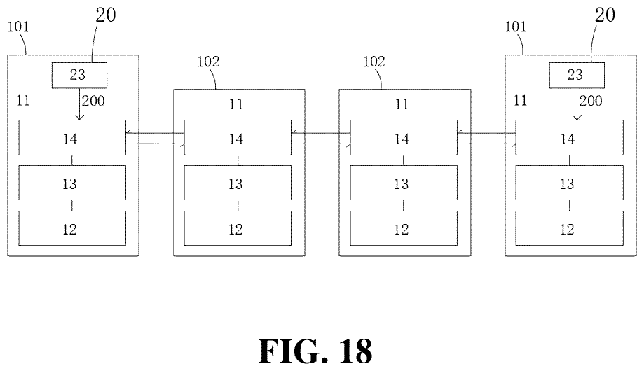

Referring to FIG. 18, in Embodiment II, each of two therapy assemblies 10 may be arranged with a voice module 23. The voice module 23 may be mounted in the host 11 of a respective one of the two therapy assemblies 10. The two therapy assemblies 10 may be named master therapy assemblies 101, and remaining therapy assemblies 10 may be slave therapy assemblies 102. The voice module 23 on each of the two master therapy assemblies 101 may only be electrically connected to the communication module 14 and control module 13 in the respective one of two master therapy assemblies 101 to transmit the user instruction. Each master therapy assembly 101 may receive the user's voice information and complete a work mode setting for the respective master therapy assembly 101. Then, the communication module 14 of the respective master therapy assembly 101 may transmit the user instruction to the other master therapy assembly 101 and the slave therapy assemblies 102, causing the other master therapy assembly 101 and the slave therapy assemblies 102 to also complete the work mode setting. That is, the slave therapy assemblies 102 may only obtain the user instruction through information interaction with the master therapy assemblies 101, while each master therapy assemblies 101 may obtain the user instruction through the voice module 23 of the respective master therapy assemblies 101 or receive the user instruction passed from the other therapy assembly 10.

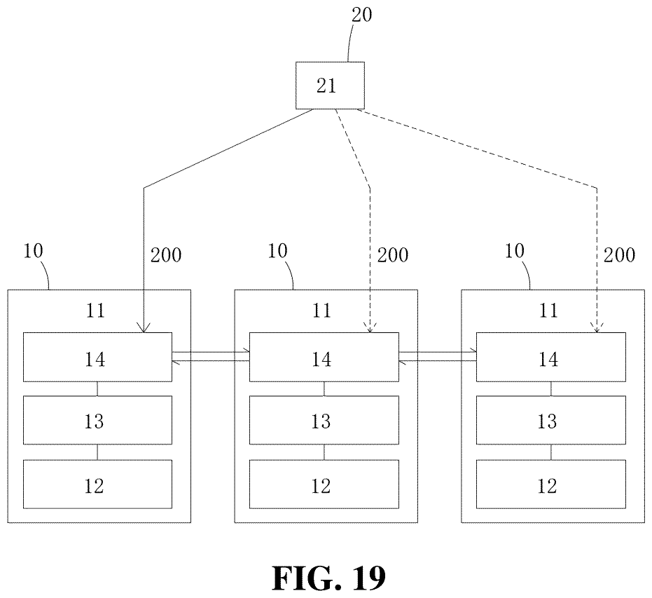

Referring to FIG. 19, in Embodiment III, at least one remote controller 21 may be provided. The remote controller 21 may send the user instruction to any therapy assembly 10. The dashed lines in FIG. 19 may indicate possible routes for the user instruction. A designated therapy assembly 10 that receives the user instruction may complete a work mode setting for the corresponding therapy assembly 10. Then, the communication module 14 of the corresponding therapy assembly 10 may transmit the user instruction to other therapy assemblies 10, causing the other therapy assemblies 10 to also complete the work mode setting. That is, each therapy assembly 10 may receive the user instruction issued by the remote controller 21 or receive the user instruction passed from other therapy assemblies 10.

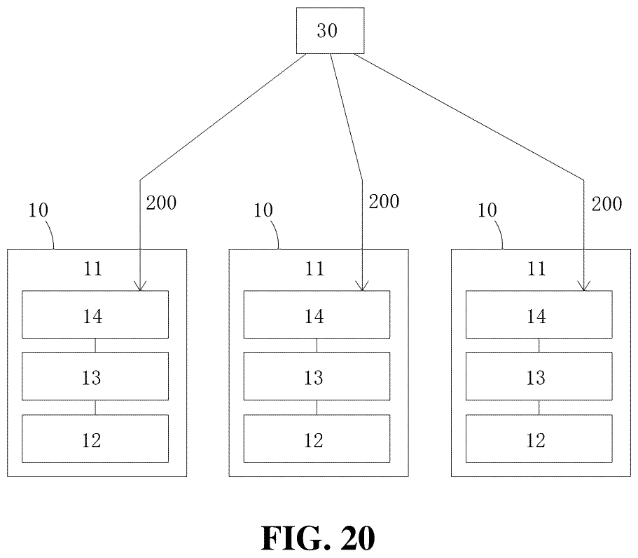

Referring to FIG. 20, in Embodiment IV, there is no instruction module 20. Instead, a smart terminal 30 may perform the function of the instruction module 20 described above. The user may send the user instruction to all therapy assemblies 10 through the smart terminal 30. Each therapy assembly 10 may receive the user instruction and complete the work mode setting of the respective therapy assembly 10. No information interaction may be required between any therapy assemblies 10.

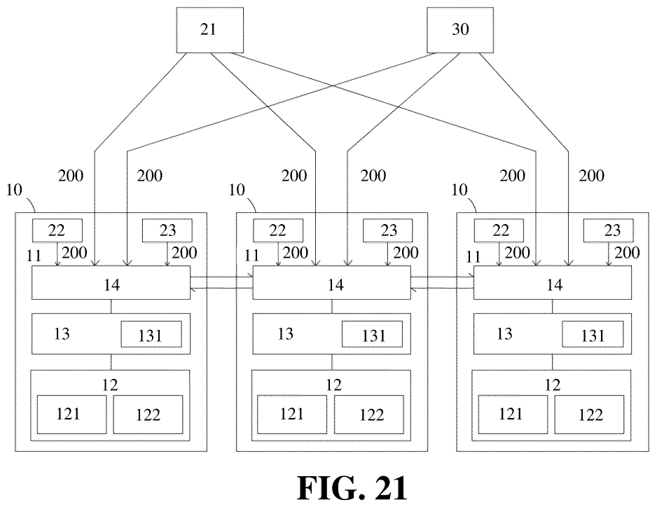

Referring to FIG. 21, in Embodiment V, at least one remote controller 21 may be provided. Each therapy assembly 10 may be arranged with a button module 22 and a voice module 23. In this case, the communication module may also receive the user instruction issued by a smart terminal. Any therapy assembly 10 may receive the user instruction and, after receiving the user instruction, complete the user instruction work mode setting for the corresponding therapy assembly 10. Then, the communication module 14 of that therapy assembly 10 may transmit the user instruction to other therapy assemblies 10, causing the other therapy assemblies 10 to also complete the work mode setting. That is, each therapy assembly 10 may receive the user instruction issued by any instruction module or smart terminal, or receive user instructions passed from other therapy assemblies 10.

All the above embodiments may achieve a synchronous control of the plurality of therapy assemblies 10, and the plurality of therapy assemblies 10 can perform cupping operation synchronously on various parts of the user's body, thereby facilitating the user to quickly perform the cupping operation using the negative pressure adsorption device.

With the aforementioned negative pressure adsorption device, the user may use the plurality of therapy assemblies 10 simultaneously to perform negative pressure adsorption therapy on various parts of the user's body at the same time, and the plurality of therapy assemblies 10 may be synchronously controlled, which is fast, convenient, and simple to operate. By controlling the plurality of therapy assemblies 10 through wireless communication, a complicated wired connection system may be simplified. When the plurality of therapy assemblies 10 are used simultaneously, there is no hassle of multiple wires, enhancing overall convenience of using the negative pressure adsorption device.

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

The disclosure claimed is:

1. A negative pressure adsorption device, comprising at least two therapy assemblies, wherein each of the at least two therapy assemblies comprises:
a host, comprising a body portion and a cup portion connected with the body portion, wherein the body portion defines an accommodation cavity;
a working module, a control module, and a communication module, mounted in the accommodation cavity, wherein the control module is electrically connected to each of the working module and the communication module, and the working module is configured to form a negative pressure in the cup portion;
wherein the communication module of each of the at least two therapy assemblies is configured to receive a user instruction; or the communication module of at least one of the at least two therapy assemblies is configured to receive the user instruction, and the communication module of one of the at least two therapy assemblies is communicatively connected to the communication module of another one of the at least two therapy assemblies; wherein the at least two therapy assemblies are capable of performing cupping operation synchronously on various parts of a user's body.

2. The negative pressure adsorption device according to claim 1, wherein the body portion comprises a cover and a housing, one of two opposite ends of the housing is connected to the cover, the cover and the housing cooperatively define the accommodation cavity, and the other of the two opposite ends of the housing is configured to be connected to the cup portion.

3. The negative pressure adsorption device according to claim 2, wherein the housing is configured to be detachably connected to the cup portion, and a cup mouth of the cup portion is an arc-shaped cup mouth, or a straight cup mouth.

4. The negative pressure adsorption device according to claim 1, wherein a diameter of a side of the body portion close to the cup portion is smaller than a diameter of a side of the body portion away from the cup portion.

5. The negative pressure adsorption device according to claim 1, wherein the communication module of only one of the at least two therapy assemblies is configured to receive the user instruction, and the communication module of one of the at least two therapy assemblies is communicatively connected to the communication module of another one of the at least two therapy assemblies.

6. The negative pressure adsorption device according to claim 1, wherein the communication module of any one of the at least two therapy assemblies is configured to receive the user instruction, and the communication module of one of any two of the at least two therapy assemblies is communicatively connected to the communication module of another one of the any two of the at least two therapy assemblies.

7. The negative pressure adsorption device according to claim 1, wherein the communication module of each of the at least two therapy assemblies is configured to simultaneously receive the user instruction, and the communication module of one of the at least two therapy assemblies is independent from the communication module of another one of the at least two therapy assemblies.

8. The negative pressure adsorption device according to claim 1, further comprising at least one instruction module, wherein the at least one instruction module is communicatively connected to the communication module of at least one of the at least two therapy assemblies.

9. The negative pressure adsorption device according to claim 8, wherein the at least one instruction module is a remote controller, and the remote controller is signally connected to the communication module of at least one of the at least two therapy assemblies; and/or
the at least one instruction module is a button module mounted on the host of at least one of the at least two therapy assemblies, and the button module is electrically connected to the communication module of the at least one of the at least two therapy assemblies and the control module of the at least one of the at least two therapy assemblies; and/or
the at least one instruction module is a voice module mounted on the host of at least one of the at least two therapy assemblies, and the voice module is electrically connected to the communication module of the at least one of the at least two therapy assemblies and the control module of the at least one of the at least two therapy assemblies.

10. The negative pressure adsorption device according to claim 9, wherein the control module is a control circuit board, each of the button module and the voice module is mounted on the control circuit board, and located on a side of the control circuit board away from the cup portion.

11. The negative pressure adsorption device according to claim 8, wherein the at least one instruction module is preset with a plurality of preset user instructions, and the plurality of preset user instructions are associated at least with a working number and a working time of the at least two therapy assemblies.

12. The negative pressure adsorption device according to claim 1, wherein the communication module of one of the at least two therapy assemblies is communicatively connected to the communication module of another one of the at least two therapy assemblies via at least one of Bluetooth, Wi-Fi, Zigbee, and RFID;
the control module is a control circuit board, the communication module is mounted on the control circuit board, and located on a side of the control circuit board away from the cup portion.

13. The negative pressure adsorption device according to claim 1, wherein each of the at least two therapy assemblies further comprises a timing unit, the control module is a control circuit board, and the timing unit is mounted on the control circuit board, and located on a side of the control circuit board away from the cup portion.

14. The negative pressure adsorption device according to claim 1, wherein the working module comprises a negative pressure unit and a heating unit, the control module is a control circuit board, and the negative pressure unit and the heating unit are mounted on the control circuit board, and located on a side of the control circuit board facing the cup portion.

15. The negative pressure adsorption device according to claim 14, wherein one or more negative pressure holes are formed in a portion of the housing connected with the cup portion to communicate the negative pressure unit with a cavity defined by the cup portion, enabling the negative pressure generated by the negative pressure unit to be transmitted through the cup portion to an area of a human body requiring therapy.

16. The negative pressure adsorption device according to claim 15, wherein the one or more negative pressure holes are formed at an end of the housing facing the cup portion and are distributed at a periphery of the heating unit.

17. The negative pressure adsorption device according to claim 14, wherein an end of the housing facing the cup portion defines a mounting hole, and the heating unit is mounted in the mounting hole and arranged at the end of the housing facing the cup portion.

18. The negative pressure adsorption device according to claim 17, wherein the heating unit comprises an infrared emitter and a light-transmitting cover; the light-transmitting cover is mounted surrounding a periphery of the infrared emitter, at least a portion of the light-transmitting cover is located on a side of the infrared emitter facing the cup portion, and the light-transmitting cover is configured to transmit an infrared light emitted by the infrared emitter toward the cup portion.

19. The negative pressure adsorption device according to claim 1, wherein the working module comprises a temperature control unit; the body portion is arranged with a metal contact piece, and the metal contact piece is mounted at an end of the body portion where the body portion is connected to the cup portion and is configured to be heated or cooled by the temperature control unit;
when the metal contact piece is used, the cup portion is removed, enabling the metal contact piece to directly contact human skin.

20. The negative pressure adsorption device according to claim 19, wherein the body portion arranged with a foam filter, the metal contact piece is a porous structure, and the foam filter is arranged on a side of the metal contact piece away from the cup portion.
